(19) United States
(12) Patent Application Publication
(10) Pub. No.: US 2021/0161783 A1
(43) Pub. Date: Jun. 3, 2021

(54) USE OF SPECIFIC BENZYLIDENE MALONATES FOR PROTECTING THE SKIN FROM CHEMICALLY INDUCED STRESS

(71) Applicant: MERCK PATENT GMBH, DARMSTADT (DE)

(72) Inventors: Marina LEFORT, Darmstadt (DE); Lilia HEIDER, Gernsheim (DE); Valerie BICARD BENHAMOU, Darmstadt (DE); Silke HORNUNG, Roedermark (DE)

(73) Assignee: MERCK PATENT GMBH, DARMSTADT (DE)

(21) Appl. No.: 17/047,264

(22) PCT Filed: Apr. 9, 2019
(86) PCT No.: PCT/EP2019/058884

§ 371 Date: Oct. 13, 2020

(30) Foreign Application Priority Data
Apr. 13, 2018 (EP) 18167261.9

(51) Int. Cl. A61K 8/37; A61Q 17/00

(52) U.S. Cl. CPC A61K 8/37; A61Q 17/00

(57) ABSTRACT

The invention relates to the use of specific benzylidenemalonates, in particular in a cosmetic preparation, for protecting the skin against chemically induced stress, in particular against chemically induced stress caused by heavy metals and/or particulate matter, or for the prevention and/or prophylaxis of epidermal morphological change.

The invention relates to the use of specific benzylidenemalonates, in particular in a cosmetic preparation, for protecting the skin against chemically induced stress, in particular against chemically induced stress caused by heavy metals and/or particulate matter, or for the prevention and/or prophylaxis of epidermal morphological change.

As the boundary layer and surface of the human body, the skin is exposed to a multiplicity of external stress factors. The human skin is an organ which protects the body against external influences by means of cell types which are specialised in a variety of ways, for example the keratinocytes, melanocytes, Langerhans cells, Merkel cells and embedded sensory cells.

The surface of the human skin is covered by a film of grease, which, depending on the given circumstances, can be regarded as an oil-in-water or water-in-oil emulsion and contains numerous active compounds, such as, for example, enzymes and vitamins. This film of grease, which is formed from the lipids secreted by sebaceous glands and keratinocytes, preserves the moisture in the skin and, as a skin barrier, protects the body against unfavourable environmental factors. This sensitive equilibrium of the skin barrier (homeostasis) can be disrupted by external or internal factors.

Amongst external factors, a differentiation should be made between external physical, chemical and biological influences on the human skin. The external physical influences include thermal and mechanical influences and the action of radiation, for example UV rays.

Physical stress factors for the skin are caused, for example, by sunlight or artificial radiation sources with a comparable spectrum, as well as compounds which may be generated by the radiation, such as undefined reactive photoproducts, which are usually free radicals or ionic.

A multiplicity of organic and inorganic UV filters and antioxidants is known which are able to absorb UV radiation and/or scavenge free radicals. They are therefore able to protect the human skin against these physical influences.

The external biological influences include, for example, the action of foreign organisms and metabolic products thereof.

External chemical influences are taken to mean, in particular, the action of substances on the skin, in particular irritants, pollutants or heavy metals. Frequent air-borne, non-allergic irritants, so-called pseudoallergens, are, for example, aerosols arising from adhesives, cleaning materials or sprays, perfume, environmental pollutants, such as, for example, aromatic hydrocarbons and/or volatile organic compounds, so-called VOCs, or also particulate matter (abbreviated to PM), such as tobacco smoke, urban dust, fine dust or particles from diesel exhausts or industrial waste gases. Particulate matter is often defined by its average size. PM 2.5 means, for example, particulate matter having an average size of 2.5 μm.

Chemical effects of this type of influence the homeostasis of the skin and generally lead to chemically induced skin irritation, in particular chemically induced by heavy metals and/or particulate matter. Possible consequences for the skin are dry, cracked or flaky skin, discolouration of the skin and/or the skin pores (for example yellowing of the skin and/or pores), itchy skin and/or sensitive skin. In case of frequent or chronic exposure to chemical irritants, skin irritations of this type can become pathological skin inflammation. Influencing of homeostasis can also be taken to mean a disruption of the barrier function of the skin. It is thought that the weaker epidermal skin barrier makes ingress of pollutants, irritants and heavy metals easier.

Chemically induced stress is therefore taken to mean the reaction of an organism or organ, for example the skin, to exposure to irritations of a chemical nature with the examples of consequences outlined above. The biochemical mechanisms triggered during chemical irritation are complex and have not yet been fully clarified.

However, it is known that heavy metals and/or particulate matter, such as tobacco smoke, urban dust, fine dust or particles from diesel exhausts or industrial waste gases, trigger oxidative stress. This triggers, for example, lipid peroxidation, which can be detected, for example, via aldehydes formed in the cell membrane. One aldehyde formed is, for example, malonic dialdehyde (MDA), as described in Janero, Free Radic Biol Med. 1990, 9(6), 515-40.

The reaction of the skin to chemical stress factors can therefore be observed, for example, from the formation of malonic dialdehyde. MDA production is therefore a suitable marker for detecting chemically induced stress of the skin.

For the purposes of the present invention, chemically induced stress is preferably taken to mean the reaction of the skin to the action of heavy metal and/or particulate matter. For the purposes of the present invention, chemically induced stress is particularly preferably taken to mean the reaction of the skin to the action of heavy metals and in particular diesel exhausts, which induce lipid peroxidation.

In order to support the skin in its natural regeneration, skin care preparations are recommended, which, besides humectants, which are intended to prevent water loss by the skin, or electrolytes, may likewise contain intercellular lipid mixtures, such as ceramides or ceramide analogues.

However, there continues to be a need for alternative active compounds which are able to protect the skin against chemically induced stress, in particular against chemically induced stress in which lipid peroxidation is induced. There continues to be a need for alternative active compounds which are able to prevent a morphological change in the skin, in particular the epidermis, after exposure to pollutants, irritants or heavy metals, in particular after exposure to heavy metals and/or particulate matter, or prevent a morphological change in the skin, in particular the epidermis.

The object of the present invention is therefore to provide an alternative active compound for non-therapeutic use, in particular in a cosmetic preparation or a medical device, which protects the skin against chemical stress factors or the consequences of the action of chemical stress factors, in particular chemical stress factors which cause lipid peroxidation. It should furthermore protect the skin, in particular the epidermis, against morphological change on exposure to chemical stress factors and maintain or restore the barrier properties of the skin.

These objects are achieved, surprisingly and in a manner which was not foreseeable by the person skilled in the art, by the use of specific benzylidenemalonates, as described below by formula I.

Benzylidenemalonates are known, for example, from U.S. Pat. No. 6,831,191 or WO 03/007906. The benzylidenemalonates described therein are described as antioxidants, which are likewise also UV absorbers (290-400 nm). It is furthermore described that these compounds are capable of stabilising other sun protection filters and therefore protecting them against decomposition due to sunlight. The compounds of the formula I, as described below, are a specific selection of benzylidenemalonates from the prior art.

An antioxidant or antioxidation agent is a chemical compound which slows or completely prevents oxidation of other substances. However, the mode of action and therefore their efficacy of the antioxidants is dependent on the nature of the molecule or radiation that is responsible for the oxidation.

Depending on their mode of action, antioxidants can be divided, for example, into free-radical scavengers, reducing agents and antioxidation synergists.

Free-radical scavengers are, for example, the natural substances tocopherol or synthetic substances, such as butylhydroxyanisole. Substances of this type form stable free radicals which do not react further and therefore lead to termination of reaction cascades.

Reducing agents are, for example, ascorbic acid or glutathione. They have a very low redox potential. Their protective action arises from them being oxidised before the substance to be protected.

Antioxidation synergists support the action of antioxidants, for example by regenerating spent antioxidants again. An example thereof is sodium EDTA.

Various test systems have therefore been identified for classifying antioxidants.

TEAC assay: With the aid of the Trolox Equivalent Antioxidative Capacity (TEAC), the antioxidative capacity of a sample can be indicated. In the measurement, the vitamin E derivative trolox serves as reference. The result is therefore quoted in trolox equivalents.

The principle of the measurement is based on the reaction of diammonium 2,2′-azinodi(3-ethylbenzothiazoline)-6-sulfonate (ABTS) with the antioxidants present in the medium to be investigated. In an oxidative medium, ABTS forms a stable, green-coloured free-radical cation, which loses its colouration on reaction with substances with an antioxidative action. Photometric measurements of the ABTS solution at 734 nm before and after a defined time span after addition of antioxidants gives an extinction difference, from which the antioxidative capacity of the substance investigated is determined by comparison with the reduction in extinction caused by trolox in various concentrations (Pellegrini, N. et al., Free Radical Biology and Medicine, 1999, 26 (9-10), 1231-1237.)

DPPH assay: In this test method, the substance to be investigated is allowed to react with the stable free radical DPPH* (2,2-diphenyl-1-picrylhydrazyl hydrate) in ethanolic solution. The reduction of DPPH* is followed via the reduction in extinction at the characteristic wavelength of the free radical. In its free-radical form, DPPH* absorbs at 515 nm. Various concentrations are investigated for each antioxidant, expressed as the ratio of moles of antioxidant to moles of DPPH*. The reduction in extinction at 515 nm is determined after 1 second, 2 minutes, 10 minutes and then every 10 minutes until the extinction remains constant. The exact initial concentration of DPPH* is determined with the aid of the extinction coefficient. The DPPH* concentration remaining is determined at each antioxidant concentration as a percentage of the initial extinction and is plotted against the molar ratio of antioxidant with DPPH*. The anti-free-radical activity is defined here as the proportion of the antioxidant that reduces the DPPH* concentration to 50 percent of the initial amount (EC₅₀).

The compound diisopropyl vanillidenemalonate (DIPVM) is described in an article published in the IFSCC Conference Proceedings, 2016, Orlando, with the title “Ease stress and support skin revitalization”, L. Heider et al, as a compound which reduces the excretion of cytokines, in particular interleukin 8, if the skin is irritated by the chemical compound phorbol 12-myristate 13-acetate (PMA). The data confirm the anti-inflammatory activity of DIPVM.

The invention relates firstly to the non-therapeutic use of compounds of the formula I

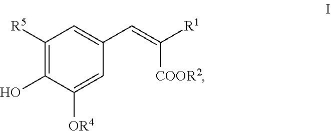

in which

R¹ denotes —C(O)CH₃ or —CO₂R³,

R², R³ and R⁴ in each case, independently of one another, denote a linear or branched alkyl group having 1 to 20 C atoms,

R⁵ denotes a linear or branched alkyl group having 1 to 20 C atoms or a linear or branched alkoxy group having 1 to 20 C atoms, for protecting the skin against chemically induced stress.

The invention furthermore relates to the use of a cosmetic preparation or a medical device comprising at least one compound of the formula I

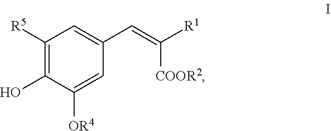

in which

R¹ denotes —C(O)CH₃ or —CO₂R³,

R², R³ and R⁴ in each case, independently of one another, denote a linear or branched alkyl group having 1 to 20 C atoms,

R⁵ denotes a linear or branched alkyl group having 1 to 20 C atoms or a linear or branched alkoxy group having 1 to 20 C atoms, for protecting the skin against chemically induced stress.

The above-mentioned definitions of chemically induced stress and its consequences, as described or preferably described above, apply.

The invention is particularly preferably directed to the non-therapeutic use of compounds of the formula I, as described above, for protecting the skin against the consequences of the action of chemical stress factors, preferably the action of pollutants, irritants and/or heavy metals which induce lipid peroxidation.

The invention furthermore relates to the non-therapeutic use of compounds of the formula I,

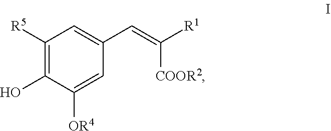

in which

R¹ denotes —C(O)CH₃ or —CO₂R³,

R², R³ and R⁴ in each case, independently of one, denote a linear or branched alkyl group having 1 to 20 C atoms,

R⁵ denotes a linear or branched alkyl group having 1 to 20 C atoms or a linear or branched alkoxy group having 1 to 20 C atoms,

for the prevention and/or prophylaxis of an epidermal morphological change.

The invention furthermore relates to the use of a cosmetic preparation or medical device comprising at least one compound of the formula I

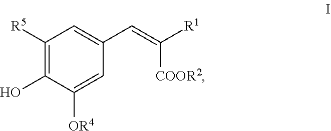

in which

R¹ denotes —C(O)CH₃ or —CO₂R³,

R², R³ and R⁴ in each case, independently of one, denote a linear or branched alkyl group having 1 to 20 C atoms,

R⁵ denotes a linear or branched alkyl group having 1 to 20 C atoms or a linear or branched alkoxy group having 1 to 20 C atoms,

for the prevention and/or prophylaxis of an epidermal morphological change.

The epidermis contains various cell types and is the part of the skin that reacts very sensitively to chemically induced stress. An epidermal morphological change can be recognised, for example, from a change in the cell nuclei, followed by separation of the dermal-epidermal interface and/or the appearance of acidophilic cytoplasm and/or from the presence of oedemas and/or from acanthosis. Modified cell nuclei are, for example, pycnotic or karyolytic cell nuclei. The compounds of the formula I preferably have a preventative action.

The invention also furthermore relates to the non-therapeutic use of compounds of the formula I

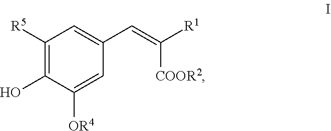

in which

R¹ denotes —C(O)CH₃ or —CO₂R³,

R², R³ and R⁴ in each case, independently of one, denote a linear or branched alkyl group having 1 to 20 C atoms,

R⁵ denotes a linear or branched alkyl group having 1 to 20 C atoms or a linear or branched alkoxy group having 1 to 20 C atoms,

for stabilising the skin barrier.

For the non-therapeutic use, the compounds of the formula I are preferably applied to the skin in cosmetic preparations or medical devices.

Accordingly, the compounds of the formula I, as described above or preferably described below, are preferably used in cosmetic preparations or medical devices for protecting the skin against chemically induced stress, in particular induced by chemical stress factors which induce lipid peroxidation, or for protecting the skin against morphological change, particularly in the epidermis, on exposure to chemical stress factors, which preferably induce lipid peroxidation.

Accordingly, the compounds of the formula I, as described above or preferably described below, are preferably used in cosmetic preparations or medical devices for stabilising the skin barrier.

The non-therapeutic uses are, in particular, cosmetic uses. Accordingly, the compounds of the formula I are employed, in particular, in cosmetic preparations.

The invention is particularly preferably directed to the non-therapeutic use of compounds of the formula I, as described above or preferably described below, if the skin comes into contact with chemical stress factors selected from the group aerosols arising from adhesives, cleaning materials or sprays, aromatic hydrocarbons and/or volatile organic compounds, heavy metals or particulate matter.

The invention is very particularly preferably directed to the non-therapeutic use of compounds of the formula I, as described above or preferably described below, if the skin comes into contact with the chemical stress factors of heavy metals, tobacco smoke, urban dust, fine dust or particles from diesel exhausts or industrial waste gases, in particular with heavy metals and particles from diesel exhausts.

As representative of the compounds of the formula I, as described above and preferably described below, the efficacy of di(2-ethylhexyl) 3,5-dimethoxy-4-hydroxybenzylidenemalonate is confirmed ex vivo in the experimental part and compared with the compound diisopropyl 5-methoxy-4-hydroxybenzylidenemalonate (DIPVM).

As described above, the substituent R¹ in compounds of the formula I can denote —C(O)CH₃ or —CO₂R³. If R¹ denotes —C(O)CH₃, the compounds of the formula I can also be called alpha-acetylcinnamic acid esters.

If R¹ denotes —CO₂R³, the compounds of the formula I are preferably called benzylidenemalonic acid esters.

Compounds of the formula I are preferably used in accordance with the invention if R¹ denotes —CO₂R³ and R³ has a meaning given above or below or a meaning preferably mentioned.

The invention therefore furthermore relates to the non-therapeutic uses of the compounds of the formula I, characterised in that the substituent R¹ in the compounds of the formula I denotes —CO₂R³ and R³ has a meaning given above or below.

As described above, the substituents R², R³ and R⁴ in each case, independently of one, denote a linear or branched alkyl group having 1 to 20 C atoms.

Straight-chain or branched alkyl groups having 1 to 4, 1 to 8, 1 to 12 or 1 to 20 C atoms conform to the formula CₚH₂ₚ₊₁ where p=1, 2, 3 or 4, or 1, 2, 3, 4, 5, 6, 7 or 8, or 1, 2, 3, 4, 5, 6, 7, 8, 9, 10, 11 or 12, or 1, 2, 3, 4, 5, 6, 7, 8, 9, 10, 11, 12, 13, 14, 15, 16, 17, 18, 19 or 20, for example methyl, ethyl, i-propyl, propyl, butyl, i-butyl or tert-butyl, pentyl, 1-, 2- or 3-methylbutyl, 1,1-, 1,2- or 2,2-dimethylpropyl, 1-ethylpropyl or hexyl, heptyl, octyl, nonyl, decyl, undecyl, dodecyl, tridecyl, tetradecyl, pentadecyl, hexadecyl, heptadecyl, octadecyl, nonadecyl or eicosyl.

If an alkyl group is not designated in greater detail, it is a straight-chain alkyl group.

Compounds of the formula I are preferably used in accordance with the invention if the substituents R² and R³ are identical.

The invention therefore furthermore relates to the non-therapeutic uses of the compounds of the formula I, characterised in that the substituents R² and R³ in the compounds of the formula I are identical.

Compounds of the formula I are preferably used in accordance with the invention if the substituents R², R³ and R⁴ in each case, independently of one, denote a linear or branched alkyl group having 1 to 8 C atoms.

The invention therefore furthermore relates to the non-therapeutic uses of the compounds of the formula I, characterised in that the substituents R², R³ and R⁴ in each case, independently of one, denote a linear or branched alkyl group having 1 to 8 C atoms.

In accordance with the invention, particular preference is given to the use of compounds of the formula I in which R¹ denotes —CO₂R³, the substituents R² and R³ are identical and denote a linear or branched alkyl group having 1 to 8 C atoms and R⁴ denotes methyl or ethyl.

In accordance with the invention, very particular preference is given to the use of compounds of the formula I in which R¹ denotes —CO₂R³, the substituents R² and R³ are identical and denote a linear or branched alkyl group having 4 to 8 C atoms and R⁴ denotes methyl.

Compounds of the formula I are preferably used in accordance with the invention if the substituent R⁵ denotes a linear or branched alkyl group having 1 to 8 C atoms or a linear or branched alkoxy group having 1 to 8 C atoms.

Compounds of the formula I are particularly preferably used in accordance with the invention if the substituent R⁵ denotes a linear or branched alkoxy group having 1 to 8 C atoms.

The invention therefore furthermore relates to the non-therapeutic uses of the compounds of the formula I, characterised in that the substituent R⁵ denotes a linear or branched alkoxy group having 1 to 8 C atoms, preferably a linear or branched alkoxy group having 1 to 4 C atoms, particularly preferably methoxy or ethoxy, very particularly preferably methoxy.

In accordance with the invention, very particular preference is given to the use of compounds of the formula I in which R¹ denotes —CO₂R³, the substituents R² and R³ are identical and denote a linear or branched alkyl group having 1 to 8 C atoms, R⁴ denotes methyl or ethyl and R⁵ denotes a linear or branched alkoxy group having 1 to 4 C atoms.

In accordance with the invention, very particular preference is given to the use of compounds of the formula I in which R¹ denotes —CO₂R³, the substituents R² and R³ are identical and denote a linear or branched alkyl group having 4 to 8 C atoms, R⁴ denotes methyl and R⁵ denotes methoxy or ethoxy.

Very particularly preferred compounds of the formula I are dimethyl 3,5-dimethoxy-4-hydroxybenzylidenemalonate, diethyl 3,5-dimethoxy-4-hydroxybenzylidenemalonate, diisopropyl 3,5-dimethoxy-4-hydroxybenzylidenemalonate, dipropyl 3,5-dimethoxy-4-hydroxybenzylidenemalonate, dibutyl 3,5-dimethoxy-4-hydroxybenzylidenemalonate, di(tert.butyl) 3,5-dimethoxy-4-hydroxybenzylidenemalonate, dipentyl 3,5-dimethoxy-4-hydroxybenzylidenemalonate, dihexyl 3,5-dimethoxy-4-hydroxybenzylidenemalonate, diheptyl 3,5-dimethoxy-4-hydroxybenzylidenemalonate, di(2-ethylhexyl) 3,4-dimethoxy-4-hydroxybenzylidenemalonate, dioctyl 3,5-dimethoxy-4-hydroxybenzylidenemalonate, dimethyl 3,5-diethoxy-4-hydroxybenzylidenemalonate, diethyl 3,5-diethoxy-4-hydroxybenzylidenemalonate, diisopropyl 3,5-diethoxy-4-hydroxybenzylidenemalonate, dipropyl 3,5-diethoxy-4-hydroxybenzylidenemalonate, dibutyl 3,5-diethoxy-4-hydroxybenzylidenemalonate, di(tert.butyl) 3,5-diethoxy-4-hydroxybenzylidenemalonate, dipentyl 3,5-diethoxy-4-hydroxybenzylidenemalonate, dihexyl 3,5-diethoxy-4-hydroxybenzylidenemalonate, diheptyl 3,5-diethoxy-4-hydroxybenzylidenemalonate, di(2-ethylhexyl) 3,5-diethoxy-4-hydroxybenzylidenemalonate, dioctyl 3,5-diethoxy-4-hydroxybenzylidenemalonate.

Of the compounds mentioned above, the compound di(2-ethylhexyl) 3,5-dimethoxy-4-hydroxybenzylidenemalonate is particularly suitable for the present invention.

The type of cosmetic preparation or medical device comprising at least one compound of the formula I, as described or preferably described above, is not restricted here.

The preparations here are usually preparations which can be used topically. The preparations in this case comprise a cosmetically or dermatologically suitable vehicle and, depending on the desired property profile, optionally further suitable ingredients. If it is a medical device, a vehicle which is tolerated for the medical device is selected.

For the purposes of the invention, “can be used topically” means that the preparation is used externally and locally, i.e. the preparation must be suitable for application, for example, to the skin.

The preparations may include or comprise, essentially consist of or consist of the said requisite or optional constituents. All compounds or components which can be used in the preparations are either known and commercially available or can be synthesised by known processes.

Suitable preparations are known, for example, from WO 03/007906.

In the non-therapeutic uses, the compounds of the formula I, as described or preferably described above, are preferably incorporated into the corresponding preparations in an amount of 0.01 to 5 percent by weight, preferably 0.05 to 2% by weight, particularly preferably in an amount of 0.08 to 0.5% by weight, where the amount indication relates to the entire amount of the preparation.

In order that the compounds of the formula I, as described or preferably described above, are able to develop their positive effect on the skin particularly well, it may be preferred to allow the compounds of the formula I to penetrate into deeper skin layers. A number of possibilities are available to this end. A preferred possibility is the use of liposomes and/or nanoemulsions comprising at least one compound of the formula I, as described above or as preferably described, in the corresponding preparation, which facilitates transport of the compounds of the formula I through the outer skin layers.

For the preparation of liposomes and/or nanoemulsions, the compounds of the formula I, as described above or as preferably described, are brought into contact with surface-active, usually amphiphilic compounds which are capable of aggregating themselves to form vesicles.

A liposome is a vesicle which encloses an aqueous phase. The vesicle consists of one or more lipid double layers. The lipid double layer consists of molecules which have both a nonpolar part and a polar part and are therefore amphiphilic. The size and structure of the liposome can vary. The liposome preferably has a diameter of about 15 to 3500 nm, particularly preferably 50 to 1000 nm. The membrane-forming molecules are usually steroids, phospholipids, glycolipids, glycosphingolipids, lipopolysaccharides, squalene, tocopherols, fatty acids or a mixture of the said molecules.

Preferred liposomes are prepared from phospholipids having a high proportion of phosphatidylcholine (greater than 80%). A synonym for phosphatidylcholine is lecithin. These are phospholipids composed of fatty acids, glycerol, phosphoric acid and choline. Lecithins are constituents of the cell membrane of animal and vegetable organisms. In general, use is made of phosphatidylcholine from the soya plant, which contains a high proportion of unsaturated, essential fatty acids, for example linoleic acid. Linoleic acid can provide the liposome membrane with high flexibility. If hydrogenated phosphatidylcholine (PC 80H) is used for the preparation of the liposome, the vesicle membrane is more rigid and lipophilic active compounds cannot be integrated into the vesicle membrane as effectively.

A nanoemulsion is characterised by a lipophilic phase which is formed from a lipid monolayer and is delimited from an aqueous phase. In this embodiment, the compounds of the formula I are in the monolayer of the membrane-forming molecules. The same comments apply to the membrane-forming molecules as described for the liposome.

Preferred solvents for the at least one compound of the formula I are, for example, alcohols, such as methanol, ethanol or isopropanol, polyols, such as glycerol, propylene glycol, butylene glycol, pentylene glycol, sorbitol, ethoxydiglycol and/or diisopropyl adipate. Particularly preferred solvents are ethanol, ethoxydiglycol and/or diisopropyl adipate.

Combination of the compounds of the formula I, as described above or as preferably described, with other active compounds which are likewise suitable for the prevention or treatment of chemically stressed skin is advantageous. This can be together in the liposome or nanoemulsion, or in the preparation intended for use.

Particularly suitable cosmetic active compounds for combination with at least one compound of the formula I, as described or preferably described above, are, for example, antireddening substances, moisturisers, humectants, antiwrinkle preparations, agents for improving the elastic properties, substances which effect stimulation of collagen synthesis, inflammation-inhibiting substances, antioxidants and/or vitamins.

Particularly suitable active compounds for combination are, for example, bisabolol, bioflavonoids, osmolytes, allantoin, biotin, 2-(4-hydroxy-3,5-dimethoxybenzyl)malonic acid bis(2-ethylhexyl) ester, marketed as RonaCare® AP by Merck, urea, niacinamide, salicylic acid or zinc oxide.

Preferred bioflavonoids are, for example, quercetin, glucosylrutin, isoquercetin, rutin or troxerutin. Preferred osmolytes are ectoin or hydroxyectoin.

Further suitable products which can be combined with the compounds of the formula I, as described above, are the products RonaCare® Poppy SE, an extract from poppies, Emblica®, an extract from the amla fruit (Indian gooseberries), RonaCare® Luremin® with the active compound 5,7-dihydroxy-2-methylchromen-4-one, RonaCare® SereneShield and RonaCare® RenouMer, an algae extract which is likewise marketed by Merck.

The following working examples are intended to explain the invention without limiting it. The invention can be carried out correspondingly throughout the range claimed. Possible variants can also be derived starting from the example.

The compound di(2-ethylhexyl) 3,5-dimethoxy-4-hydroxy-benzylidenemalonate was prepared in accordance with Example XVII of WO 03/007906 and employed in the following example.

The compound diisopropyl 3-methoxy-4-hydroxy benzylidenemalonate (DIPVM) was prepared in accordance with Example VI of EP 1952843 and employed in the following example.

### EXAMPLE 1: EX-VIVO STUDY ON LIVING HUMAN SKIN EXPLANTS

An ex-vivo study was carried out on skin explants from a 55-year-old woman with white skin colour (P1922-AB54). The explants are kept in a functioning state in a BEM culture medium (explants medium from BIO-EC) at 37° C. in a moist atmosphere containing 5% of CO₂. 4 explants are used in each case in an experimental series of a substance, the control or the reference.

The irritant employed to trigger chemically induced stress is a solution of metals and heavy metals, purchased from Merck under article number 1.10714.0500, containing, inter alia, Al, As, B, Ba, Be, Ca, Cd, Cr, Cu, Fe, Hg, K, Li, Mg, Mn, Na, Ni, P, Pb, Sa, Sc, Se, Sr, Te, Ti, Y, Zn, where 0.1% of diesel particles were also added to this solution (1 mg/ml, particle size PM2.5, 1650b from NIST).

Compounds A and B to be investigated are employed in 0.5% solution in ethanol. The influence of ethanol on the explant is investigated in parallel (compound C=ethanol). The reference employed is the gel from DECLÉOR with the article number 56300 (gel D; decléor gelée hydratante anti-pollution), which is known for its protection of the skin against chemicals, heavy metals and diesel particles. The control used is an untreated sample which only comes into contact with the culture medium.

Compound A is diethylhexyl syringlyidenemalonate (DESM, di(2-ethylhexyl) 3,5-dimethoxy-4-hydroxybenzylidenemalonate).

Compound B is diisopropyl vanilidene malonate (DIPVM, diisopropyl 3-methoxy-4-hydroxybenzylidenemalonate).

Irritant from Merck with the article number 1.10714.0500, where the metals/heavy metals are dissolved in 5% hydrochloric acid, with the following concentrations,

Al 0.01 mg/ml

As 0.01 mg/ml

B 0.001 mg/ml

Ba 0.001 mg/ml

Be 0.0005 mg/ml

Ca 0.005 mg/ml

Cd 0.001 mg/ml

Cr 0.001 mg/ml

Cu 0.001 mg/ml

Fe 0.001 mg/ml

Hg 0.0025 mg/ml

K 0.0495 mg/ml

Li 0.001 mg/ml

Mg 0.0005 mg/ml

Mn 0.0005 mg/ml

Na 0.01 mg/ml

Ni 0.0025 mg/ml

P 0.005 mg/ml

Pb 0.01 mg/ml

Sc 0.0005 mg/ml

Sa 0.01 mg/ml

Sr 0.0005 mg/ml

Te 0.01 mg/ml

Ti 0.001 mg/ml

Y 0.0005 mg/ml

Zn 0.001 mg/ml.

with diesel particles being added, as described above.

Sample Application

The explants are treated once daily for 4 days (day 0=D0, day 1=D1, day 2=D2 and day 3=D3) with the solutions to be tested containing compound A and B as well as compound C and gel D (2 μl per explant corresponding to 2 mg/cm²). On D3, the solutions to be tested and the reference are treated 4 hours before application of the irritant.

Explants with compound C are in each case left to dry for 10 minutes in order that the ethanol can evaporate.

All explants remain in culture medium, half of which is replenished on D2 and all of which is replenished on D3.

On D3, the explants are treated with the irritant containing the metals/heavy metals and diesel particles, as described above, where a paper disc with a diameter of 9 mm is soaked with 30 μl of the irritant. The irritant acts on the respective explant for 24 hours.

On day 4 (D4), three explants are collected for each of the compounds to be investigated. Each explant is divided, and one part is frozen at −80° C., the second half is fixed in a buffered formol solution (formaldehyde solution). The fourth explant remains frozen at −80° C.

The culture medium of the treated explants is investigated for the compound MDA. MDA stands for malonic dialdehyde (propanedial) and is a known marker for oxidative stress, as described above.

Histological Investigation:

The explants fixed for 24 hours are dehydrogenated and impregnated in paraffin. Areas with a thickness of 5 μm are then cut and investigated under the microscope (for example Leica DMLB or Olympus BX43). Corresponding images are produced, using the Masson staining method (Masson's trichrome staining, Goldner variant), a standard colouring method in histology. The staining allows cells and surrounding tissue to be differentiated.

The changes in the explants with respect to the stratum corneum, the epidermis, the dermal-epidermal threshold and the papillary dermis thereby become visible.

The epidermis contains various cell types and is the part of the skin that reacts very sensitively to chemically induced stress. The effect of the irritant in the study is evident, for example, from the epidermal morphology. The change in epidermal morphology can be recognised, for example, from a change in the cell nuclei, separation of the dermal-epidermal interface and/or the appearance of acidophilic cytoplasm and/or from the presence of oedemas. Modified cell nuclei are, for example, pycnotic cell nuclei or karyolytic cell nuclei.

MDA Investigation:

The culture medium of each investigated explant is mixed with a salt medium (HBBS Medium Hank's Balanced Salts) and a TBAR solution and heated in a water bath at 80° C. for 15 minutes. The TBAR solution contains thiobarbituric acid, hydrochloric acid and trichloroacetic acid. Many substances which are unrelated to the lipoperoxidation react with the thiobarbituric acid, for example glucose. After cooling, malonic dialdehyde (MDA) is extracted with butanol in a liquid/liquid extraction, and the then investigated by spectrofluorimetry (excitation 515 nm, emission: 550 nm). The MDA concentration indicated below is quoted in nmol/l.

Test Results:

1. For interpretation of the results, it is taken into account that the culture medium of the untreated explants (without irritant and without compounds A, B, C and gel D) also contains MDA on day 4.

For analysis of the results, this amount of MDA present on average in the untreated explants is therefore subtracted from the values obtained for the explant samples in order to determine the influence of MDA production owing to the irritant.

The investigation of ethanol (compound C) under chemical stress with the irritant shows that ethanol has no influence on the target molecule MDA. The results of the investigations of compounds A and B in ethanolic solution are therefore effects of the compound and not of the solvent.

The MDA concentration in the culture medium of the untreated sample is on average 98.3 nmol/l.

The MDA concentration in the culture medium of the explant treated only with the irritant is on average 143.6 nmol/l.

The average MDA concentration atributable to the irritant alone is 45.3 nmol/l (143.6−98.3=45.3 [nmol/l]).

The MDA concentration in the culture medium of the explant treated with compound A and the irritant is on average 129.0 nmol/l.

The MDA concentration in the culture medium of the explant which has been treated without irritant, but with compound A is on average 94.7 nmol/l.

The average MDA concentration in the presence of compound A which is atributable to the irritant alone is 34.3 nmol/l (129.0−94.7=34.3 [nmol/l]). The MDA concentration in the culture medium of the explant treated with compound B and the irritant is on average 135.5 nmol/l.

The MDA concentration in the culture medium of the explant which has been treated without irritant, but with compound B is on average 93.6 nmol/l.

The average MDA concentration in the presence of compound B which is atributable to the irritant alone is 41.9 nmol/l (135.5−93.6 nmol/l=41.9 [nmol/l]).

The MDA concentration in the culture medium of the explant treated with gel D and irritant is on average 145.0 nmol/l.

The MDA concentration in the culture medium of the explant which has been treated without irritant, but with gel D is on average 96.0 nmol/l.

The average MDA concentration in the presence of gel D which is atributable to the irritant alone is 49.0 nmol/l (145.0−96.0=49.0 [nmol/l]).

Interpretation of the Results:

The ethanolic solution containing compound A induces 10% lower MDA production, which corresponds to a significant reduction. If this is standardised to the amount of MDA released only by the irritant, the solution containing compound A hinders MDA production by 24%

The ethanolic solution containing compound B induces 6% lower MDA production, which corresponds to an insignificant reduction. If this is standardised to the amount of MDA released only by the irritant, the solution containing compound B hinders MDA production by 8%.

The reference containing gel D has no significant effect (1% change).

2. Interpretation of the Histological Results

The histological investigation shows that explants treated with compound C (ethanol) and the irritant exhibits a clear morphological change in the epidermis.

FIG. 1 shows a histological picture of the untreated explant. FIG. 2 shows the epidermal morphological change in the explant on treatment with ethanol and irritant. The epidermis in FIG. 2 shows numerous pycnotic cells with perinuclear oedemas in the upper epidermis and clear spongiosis in the basal layer with few zones with acanthosis.

The histologic investigation shows that explants treated with compound A and the irritant, as described above and shown in FIG. 3, have no epidermal change compared with the untreated sample (FIG. 1).

The histological investigation shows that explants treated with compound B and the irritant, as described above and shown in FIG. 4, have an epidermal morphological change compared with the untreated sample (FIG. 1). The epidermis in FIG. 4 shows numerous pycnotic cells with perinuclear oedemas in the upper epidermis.

The histological investigation shows that explants treated with gel D and the irritant, as described above and shown in FIG. 5, have an epidermal morphological change compared with the untreated sample (FIG. 1).

The epidermis in FIG. 5 shows some pycnotic cells with perinuclear oedemas in the upper epidermis.

If the morphological epidermal change is quantified by assigning a score to the detectable changes, as described above, the change can be compared with one another. For the histological investigations indicated, this would mean that, compared with the untreated control and the treated explant with compound A and the irritant, the condition of the epidermis worsens by about 55% in explants treated with compound B and the irritant or in explants treated with ethanol and the irritant.

For the histological investigations indicated, this would mean that, compared with the untreated control and the treated explant with compound A and the irritant, the condition of the epidermis worsens by about 33% in explants treated with the reference and the irritant.

### LIST OF FIGURES

FIG. 1 shows a histological picture of the untreated explant.

FIG. 2 shows a histological picture of the explant treated with ethanol and irritant.

FIG. 3 shows a histological picture of the explant treated with the solution containing compound A and irritant.

FIG. 4 shows a histological picture of the explant treated with the solution containing compound B and irritant.

FIG. 5 shows a histological picture of the explant treated with gel D and irritant.

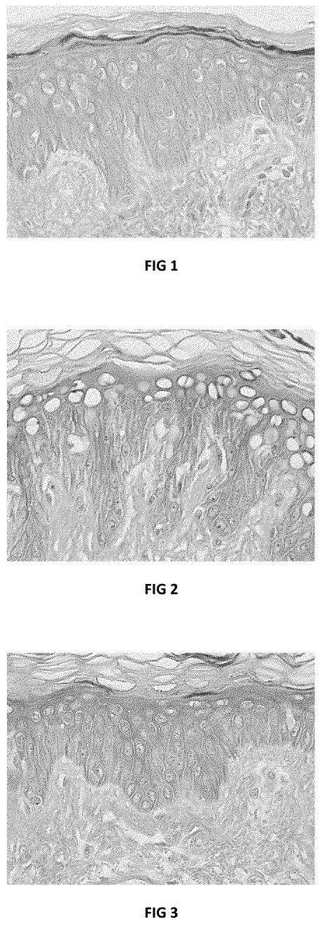

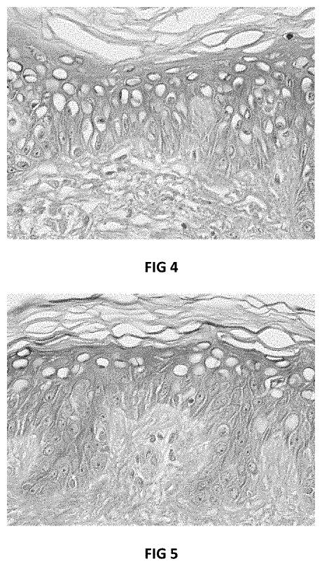

1. A method
for protecting the skin against chemically induced stress or for stabilising the skin barrier,
for the prevention and/or prophylaxis of an epidermal morphological change, comprising administering to the skin a compound of formula I 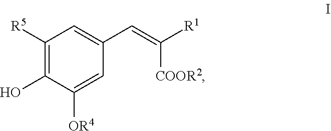
in which
R¹ denotes —C(O)CH₃ or —CO₂R³,
R², R³ and R⁴ in each case, independently of one, denote a linear or branched alkyl group having 1 to 20 C atoms,
R⁵ denotes a linear or branched alkyl group having 1 to 20 C atoms or a linear or branched alkoxy group having 1 to 20 C atoms.

2. A method for the prevention and/or prophylaxis of an epidermal morphological change according to claim 1, comprising administering to the epidermis a compound of formula formula I, 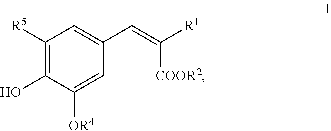
in which
R¹ denotes —C(O)CH₃ or —CO₂R³,
R², R³ and R⁴ in each case, independently of one, denote a linear or branched alkyl group having 1 to 20 C atoms,
R⁵ denotes a linear or branched alkyl group having 1 to 20 C atoms or a linear or branched alkoxy group having 1 to 20 C atoms.

3. A method for protecting the skin against chemically induced stress or for stabilising the skin barrier according to claim 1, wherein the compound of formula I is in a cosmetic preparation or composition comprising a cosmetically acceptable carrier and the at least one compound of formula I 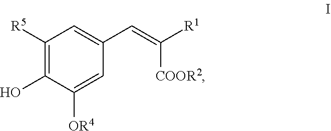
in which
R¹ denotes —C(O)CH₃ or —CO₂R³,
R², R³ and R⁴ in each case, independently of one, denote a linear or branched alkyl group having 1 to 20 C atoms,
R⁵ denotes a linear or branched alkyl group having 1 to 20 C atoms or a linear or branched alkoxy group having 1 to 20 C atoms.

4. A method for the prevention and/or prophylaxis of an epidermal morphological change according to claim 1, wherein the compound of formula I is in a cosmetic preparation or composition comprising a cosmetically acceptable carrier and the at least one compound of formula I 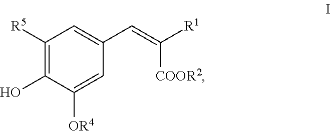
in which
R¹ denotes —C(O)CH₃ or —CO₂R³,
R², R³ and R⁴ in each case, independently of one, denote a linear or branched alkyl group having 1 to 20 C atoms,
R⁵ denotes a linear or branched alkyl group having 1 to 20 C atoms or a linear or branched alkoxy group having 1 to 20 C atoms.

5. A method for protecting the skin against chemically induced stress or for stabilising the skin barrier according to claim 1, wherein the compound of formula I is in a medical device comprising at least one compound of formula I 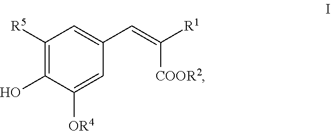
in which
R¹ denotes —C(O)CH₃ or —CO₂R³,
R², R³ and R⁴ in each case, independently of one, denote a linear or branched alkyl group having 1 to 20 C atoms,
R⁵ denotes a linear or branched alkyl group having 1 to 20 C atoms or a linear or branched alkoxy group having 1 to 20 C atoms.

6. A method for the prevention and/or prophylaxis of an epidermal morphological change according to claim 1, wherein the compound of formula I is in a medical device comprising at least one compound of formula I 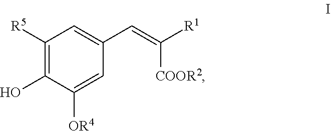
in which
R¹ denotes —C(O)CH₃ or —CO₂R³,
R², R³ and R⁴ in each case, independently of one, denote a linear or branched alkyl group having 1 to 20 C atoms,
R⁵ denotes a linear or branched alkyl group having 1 to 20 C atoms or a linear or branched alkoxy group having 1 to 20 C atoms.

7. The method according to claim 1, wherein R¹ denotes —CO₂R³.

8. The method according to claim 1, wherein R² and R³ are identical.

9. The method according to claim 1, wherein R², R³ and R⁴ in each case, independently of one, denote a linear or branched alkyl group having 1 to 8 C atoms.

10. The method according to claim 1, wherein R⁵ denotes a linear or branched alkoxy group having 1 to 8 C atoms.

11. A method for protecting the skin against chemically induced stress or for stabilising the skin barrier according to claim 1, comprising administering to the skin a compound of formula I 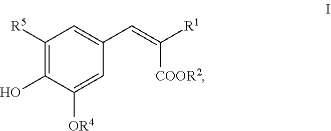
in which
R¹ denotes —C(O)CH₃ or —CO₂R³,
R², R³ and R⁴ in each case, independently of one, denote a linear or branched alkyl group having 1 to 20 C atoms,
R⁵ denotes a linear or branched alkyl group having 1 to 20 C atoms or a linear or branched alkoxy group having 1 to 20 C atoms.

12. A method according to claim 1, wherein administration is topical.

13. A method according to claim 1, wherein administration is external and local.
